(19) United States
(12) Patent Application Publication
(10) Pub. No.: US 2016/0258937 A1
(43) Pub. Date: Sep. 8, 2016

(54) URINE TEST DEVICE INCORPORATING URINARY CATHETER OR URINARY BAG ATTACHMENT DEVICE AND ASSOCIATED METHOD OF USE

(71) Applicants: J. Kent ELLINGTON, Charlotte, NC (US); Tracey Lee ELLINGTON, Charlotte, NC (US)

(72) Inventors: J. Kent ELLINGTON, Charlotte, NC (US); Tracey Lee ELLINGTON, Charlotte, NC (US)

(21) Appl. No.: 14/828,601

(22) Filed: Aug. 18, 2015

Related U.S. Application Data
(60) Provisional application No. 62/129,064, filed on Mar. 6, 2015.

(51) Int. Cl. G01N 33/52; A61M 25/00; A61M 39/10; B01L 3/00

(52) U.S. Cl. CPC G01N 33/528; B01L 3/5023; A61M 25/0017; A61M 39/10; B01L 2300/0609; B01L 2300/0825; B01L 2200/025; A61B 2562/0295; A61M 2230/201

(57) ABSTRACT

The present invention provides a urine test device incorporating a urinary catheter attachment device. This urine test device may be used in an ER environment or the like to simultaneously test a catheterized (or uncatheterized) patient for pregnancy, alcohol, a variety of illicit and legal drugs, blood in the urine, urine glucose, disease, point of care concerns, and/or the like. The urine test device is selectively attached to a patient's urinary catheter (or the associated urinary bag or tubing) to conveniently extract a urine sample for testing on site. Alternatively, a urine sample can be introduced into the urine test device via a more conventional approach, such as voluntarily by the patient, or by syringe by a medical professional. Appropriate attachments are provided for these purposes.

### CROSS-REFERENCE TO RELATED APPLICATION

The present patent application/patent claims the benefit of priority of co-pending U.S. Provisional Patent Application No. 62/129,064, filed on Mar. 6, 2015, and entitled “FOLEY URINE TEST SYSTEMS AND METHODS,” the contents of which are incorporated in full by reference herein.

### FIELD OF THE INVENTION

The present invention relates generally to the medical field. More specifically, the present invention relates to a urine test device incorporating a urinary catheter attachment device. This urine test device may be used in an emergency room (ER) environment or other clinical setting in which urinalysis is indicated to simultaneously test a catheterized (or uncatheterized) patient for pregnancy, alcohol, a variety of illicit and legal drugs, blood in the urine, urine glucose, disease, point of care concerns, and/or the like.

### BACKGROUND OF THE INVENTION

A variety of conventional urine test devices exist to test for pregnancy, alcohol, a variety of illicit and legal drugs, blood in the urine, urine glucose, disease, point of care concerns, and/or the like, either individually or simultaneously. All of these urine test devices work the same way—a patient voluntarily or involuntarily submits a urine sample to which the associated test strip(s) or the like are exposed, such as in a cup or other housing. The test strip(s) typically detect a substance or metabolite in the urine via chemical reaction with a reagent and/or antigen on the test strip(s), providing a visual indicator in a few minutes time. This is the easiest and most established method of testing for pregnancy, alcohol, a variety of illicit and legal drugs, blood in the urine, urine glucose, disease, point of care concerns, and/or the like, and is generally preferred over saliva, hair, and blood testing, optionally using gas chromatography/mass spectrometry, as these may take more time and be more expensive.

An issue that still remains, however, is what to do with an incapacitated and/or catheterized patient in an ER environment or the like. To date, a urine sample must be collected from such patients manually by a medical professional, such as by extraction from a urinary bag or tubing using a syringe, and then introduced into the conventional urine test device used. This is a time consuming, cumbersome, and inefficient process, prone to spillage, loss of sample, contamination, and errors in labeling. Often, multiple personnel must obtain multiple samples. A solution to this problem is still needed in the art.

### BRIEF SUMMARY OF THE INVENTION

In various exemplary embodiments, the present invention provides a urine test device incorporating a urinary catheter attachment device. This urine test device may be used in an ER environment or the like to simultaneously test a catheterized (or uncatheterized) patient for pregnancy, alcohol, a variety of illicit and legal drugs, blood in the urine, urine glucose, disease, point of care concerns, and/or the like. The urine test device is selectively attached to a patient's urinary catheter (or the associated urinary bag or tubing) to conveniently extract a urine sample for testing on site. Alternatively, a urine sample can be introduced into the urine test device via a more conventional approach, such as voluntarily by the patient, or by syringe by a medical professional. Appropriate attachments are provided for these purposes.

In one exemplary embodiment, the present invention provides a urine test device, including: a housing including one or more internal chambers in fluid communication with an inlet port; one or more urine test strips disposed within the one or more internal chambers of the housing; and an attachment device coupled to the inlet port, wherein the attachment device is operable for selectively coupling the inlet port to a urine source from which urine is introduced into the one or more internal chambers of the housing and exposed to the one or more urine test strips. The housing further includes one or more substantially transparent windows through which one or more visual indicators associated with the one or more urine test strips are selectively read by a user. The housing further includes one or more urine test labels associated with the one or more urine test strips. The one or more urine test strips are configured to test for one or more of pregnancy, alcohol, an illicit or legal drug, blood in the urine, urine glucose, disease, and a point of care concern. Optionally, the attachment device includes a lure lock that is selectively coupled to one of a urinary catheter and a urinary bag or tubing. Optionally, the attachment device further includes a conduit coupling the lure lock to the housing. Alternatively, the attachment device includes one of a funnel structure and a syringe attachment structure. Optionally, the attachment device further includes a conduit coupling the one of the funnel structure and the syringe attachment structure to the housing. The urine test device further includes a drain mechanism in fluid communication with the one or more internal chambers of the housing operable for selectively draining urine from the housing.

In another exemplary embodiment, the present invention provides a presumptive urine test method, including: providing a urine test device, including: a housing including one or more internal chambers in fluid communication with an inlet port; one or more urine test strips disposed within the one or more internal chambers of the housing; and an attachment device coupled to the inlet port, wherein the attachment device is operable for selectively coupling the inlet port to a urine source from which urine is introduced into the one or more internal chambers of the housing and exposed to the one or more urine test strips; coupling the inlet port to the urine source using the attachment device, introducing urine into the one or more internal chambers of the housing, and exposing the urine to the one or more urine test strips; and reading one or more visual indicators associated with the one or more urine test strips. The housing further includes one or more substantially transparent windows through which the one or more visual indicators associated with the one or more urine test strips are read. The housing further includes one or more urine test labels associated with the one or more urine test strips. The one or more urine test strips are configured to test for one or more of pregnancy, alcohol, an illicit or legal drug, blood in the urine, urine glucose, disease, and a point of care concern. Optionally, the attachment device includes a lure lock that is selectively coupled to one of a urinary catheter and a urinary bag or tubing. Optionally, the attachment device further includes a conduit coupling the lure lock to the housing. Alternatively, the attachment device includes one of a funnel structure and a syringe attachment structure. Optionally, the attachment device further includes a conduit coupling the one of the funnel structure and the syringe attachment structure to the housing. The urine test device further includes a drain mechanism in fluid communication with the one or more internal chambers of the housing operable for selectively draining urine from the housing.

### BRIEF DESCRIPTION OF THE DRAWINGS

The present invention is illustrated and described herein with reference to the various drawings, in which like reference numbers are used to denote like device components/method steps, as appropriate, and in which:

FIG. 1 is a front perspective view of one exemplary embodiment of the urine test device of the present invention;

FIG. 2 is a rear perspective view of one exemplary embodiment of the urine test device of the present invention; and

FIG. 3 is a flow chart of one exemplary embodiment of the urine test method of the present invention.

### DETAILED DESCRIPTION OF THE INVENTION

Again, in various exemplary embodiments, the present invention provides a urine test device incorporating a urinary catheter attachment device. This urine test device may be used in an ER environment or the like to simultaneously test a catheterized (or uncatheterized) patient for pregnancy, alcohol, a variety of illicit and legal drugs, blood in the urine, urine glucose, disease, point of care concerns, and/or the like. The urine test device is selectively attached to a patient's urinary catheter (or the associated urinary bag or tubing) to conveniently extract a urine sample for testing on site. Alternatively, a urine sample can be introduced into the urine test device via a more conventional approach, such as voluntarily by the patient, or by syringe by a medical professional. Appropriate attachments are provided for these purposes.

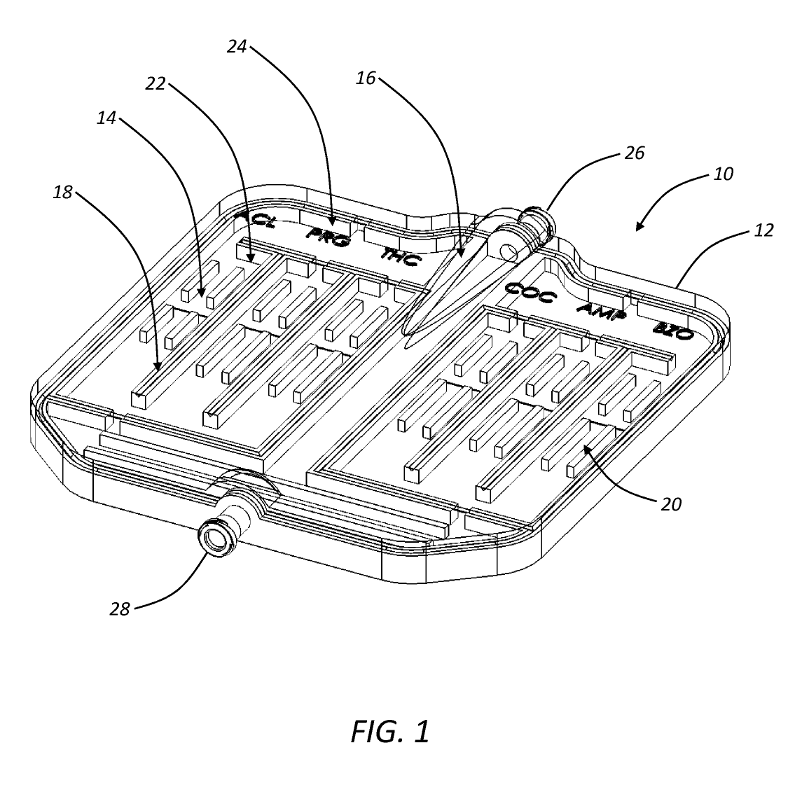

Referring now specifically to FIG. 1, in one exemplary embodiment, the urine test device 10 of the present invention includes a housing 12 defining one or more internal chambers 14 in fluid communication with an inlet port 16. In the exemplary embodiment illustrated, the housing is a thin rectangular prismatic structure that defines six internal chambers 14 arranged essentially in columns. It will be readily apparent to those of ordinary skill in the art, however, that other suitable configurations may be utilized. For example, fewer or more internal chambers 14 could be arranged in a concentric manner within a cylindrical housing 12, etc. In general, the internal chambers 14 are sized such that they are each capable of containing a predetermined volume of urine to be tested, this urine received from the common inlet port 16, which may simply be an opening manufactured into the exterior of the housing 12, a fluid channel running between the various internal chambers 14, a defined port structure, etc. The internal chambers 14 may be separate chambers, or they may simply be separate portions of the same unitary internal chamber 14, with or without internal divider walls 18. As a whole, the housing 12 and its associated components are manufactured from any material that is suitable for medical equipment exposed to bodily fluids, such as a plastic material. It should be noted that the housing 12 may be a unitary structure or consist of multiple components.

One or more urine test strips 20 are disposed within the one or more internal chambers 14 of the housing 12. The one or more urine test strips 20, well known to those of ordinary skill in the art, are configured to test for one or more of pregnancy (PRG), alcohol (ACL), an illicit or legal drug, blood in the urine, urine glucose, a disease, and a point of care concern. For example, the drug test strips 20 may be used to test for any or all of the following non-limiting exemplary substances:

Nicotine (NIC)

Amphetamines (AMPHs)

Barbiturates (BARs)

Benzodiazepines (BZOs)

Buprenorphine (Suboxone)

Cocaine (COC)

Ecstacy (MDMA)

Marijuana (THC)

Methamphetamines (MAMPs)

Methadone (MTD)

Phencyclidine (PCP)

Propoxyphene (PPX)

Opiates (OPI)

Oxycodone (OXY)

Trycyclic Antidepressants (TCAs)

The one or more urine test strips 20 typically detect a substance or metabolite in the urine via chemical reaction with a reagent and/or antigen on the test strip(s), providing a visual indicator in a few minutes time. Accordingly, the housing 12 includes one or more substantially transparent windows 22 through which one or more visual indicators associated with the one or more urine test strips 20 are selectively read by a user. The housing 12 also includes one or more urine test labels 24 associated with the one or more urine test strips 20, thereby allowing the type/subject of each test to be easily visually identified.

An attachment device 26 is directly or indirectly coupled to the inlet port 16. The attachment device 26 is operable for coupling the inlet port 16 to a urine source (such as a urinary catheter or urinary bag or tubing, not illustrated) from which urine is introduced into the one or more internal chambers 14 of the housing 12 and exposed to the one or more urine test strips 20. Optionally, the attachment device 26 includes a one-way lure lock check valve or the like that is directly or indirectly coupled to the urinary catheter or urinary bag or tubing. Optionally, the attachment device 26 also includes a conduit or the like coupling the lure lock to the housing. This arrangement allows urine to flow out of the urinary catheter or urinary bag or tubing into the housing 12, but does not allow urine to flow out of the housing 12 into the urinary catheter or urinary bag or tubing. Alternatively, the attachment device 26 includes a funnel structure (not illustrated) or a syringe attachment structure (not illustrated). This allows a urine sample to be introduced into the urine test device 10 via a more conventional approach, such as voluntarily by the patient, or by syringe by a medical professional. In general, urine flows into the housing 12 by the operation of gravity.

The urine test device 10 further includes a drain mechanism 28 in fluid communication with the one or more internal chambers 14 of the housing 12 operable for selectively draining urine from the housing 12.

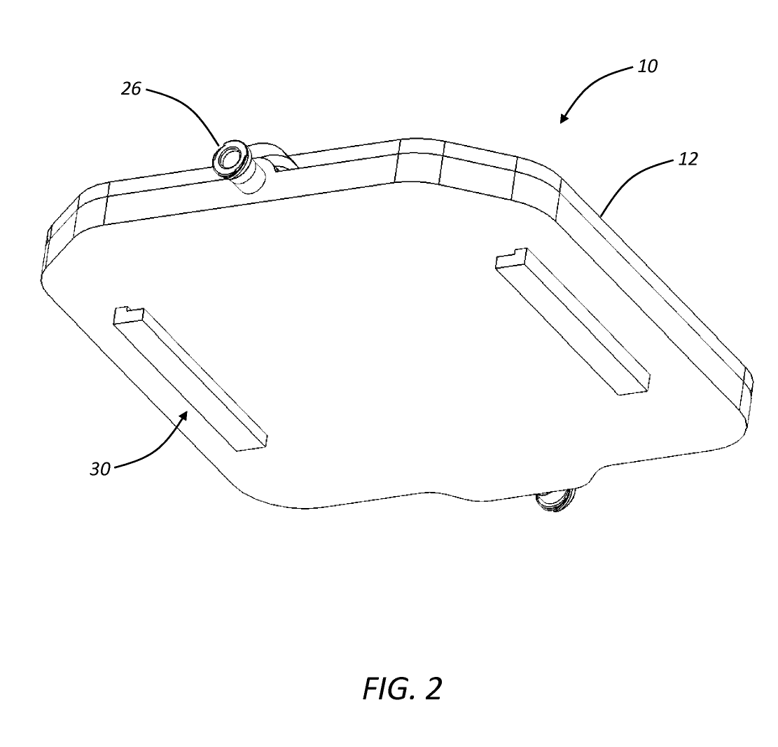

Referring now specifically to FIG. 2, in one exemplary embodiment, the top, bottom, side, or back of the housing 12 further includes one or more attachment structures 30 for removably attaching the housing to a urinary bag or tubing, a urinary bag support, a patient's bed, or any other suitable structure during use. These one or more attachment structures 30 may consist of molded structures, elastic bands, hooks, clasps, etc.

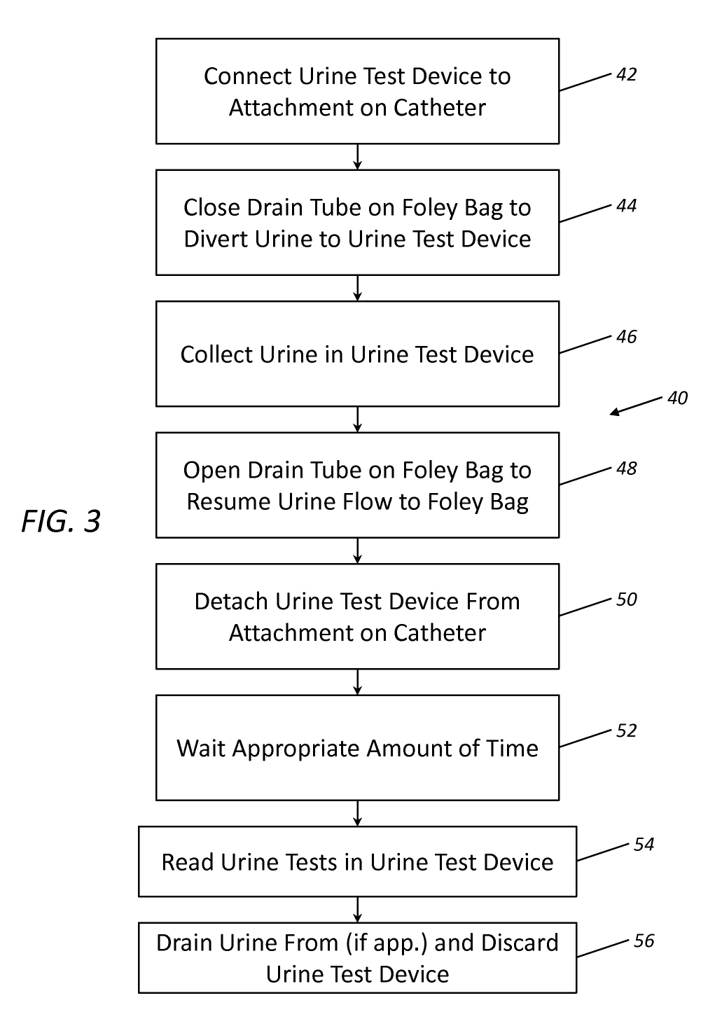

Referring now specifically to FIG. 3, in one exemplary embodiment, the presumptive urine test method 40 of the present invention includes first connecting the attachment device 26 (FIGS. 1 and 2) of the urine test device 10 (FIGS. 1 and 2) to a corresponding attachment device on a urinary catheter or urinary bag or tubing that is in use (Block 42). The urine test method 40 next includes closing the drain tube on the urinary bag by pinching it off or otherwise to divert the flow of urine to the urine test device 10 (Block 44). It should be noted that the urine test device 10 may include a drain tube or the like that is coupled to the drain tube on the urinary bag. The urine test device 10 is then filled to the required degree (Block 46). The urine test method 40 next includes opening the drain tube on the urinary bag to divert the flow of urine back to the urinary bag (Block 48), at which point the urine test device 10 is detached from the attachment device on the urinary catheter or urinary bag or tubing (Block 50). After an appropriate period of time passes for the required reactions to occur (Block 52), the urine test strips in the urine test device 10 are read (Block 54). Finally, the urine is drained from the urine test device 10 (if it is not drained before reading) and the urine test device 10 is discarded as is other medical waste (Block 56).

Although the present invention is illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present invention, are contemplated thereby, and are intended to be covered by the following non-limiting claims.

What is claimed is:

1. A urine test device, comprising:
a housing comprising one or more internal chambers in fluid communication with an inlet port;
one or more urine test strips disposed within the one or more internal chambers of the housing; and
an attachment device coupled to the inlet port, wherein the attachment device is operable for selectively coupling the inlet port to a urine source from which urine is introduced into the one or more internal chambers of the housing and exposed to the one or more urine test strips.

2. The urine test device of claim 1, wherein the housing further comprises one or more substantially transparent windows through which one or more visual indicators associated with the one or more urine test strips are selectively read by a user.

3. The urine test device of claim 1, wherein the housing further comprises one or more urine test labels associated with the one or more urine test strips.

4. The urine test device of claim 1, wherein the one or more urine test strips are configured to test for one or more of pregnancy, alcohol, an illicit or legal drug, blood in the urine, urine glucose, a disease, and a point of care concern.

5. The urine test device of claim 1, wherein the attachment device comprises a lure lock that is selectively coupled to one of a urinary catheter and a urinary bag or tubing.

6. The urine test device of claim 5, wherein the attachment device further comprises a conduit coupling the lure lock to the housing.

7. The urine test device of claim 1, wherein the attachment devices comprises one of a funnel structure and a syringe attachment structure.

8. The urine test device of claim 7, wherein the attachment device further comprises a conduit coupling the one of the funnel structure and the syringe attachment structure to the housing.

9. The urine test device of claim 1, further comprising a drain mechanism in fluid communication with the one or more internal chambers of the housing operable for selectively draining urine from the housing.

10. A urine test method, comprising:
providing a urine test device, comprising:
a housing comprising one or more internal chambers in fluid communication with an inlet port;
one or more urine test strips disposed within the one or more internal chambers of the housing; and
an attachment device coupled to the inlet port, wherein the attachment device is operable for selectively coupling the inlet port to a urine source from which urine is introduced into the one or more internal chambers of the housing and exposed to the one or more urine test strips;
coupling the inlet port to the urine source using the attachment device, introducing urine into the one or more internal chambers of the housing, and exposing the urine to the one or more urine test strips; and
reading one or more visual indicators associated with the one or more urine test strips.

11. The urine test method of claim 10, wherein the housing further comprises one or more substantially transparent windows through which the one or more visual indicators associated with the one or more urine test strips are read.

12. The urine test method of claim 10, wherein the housing further comprises one or more urine test labels associated with the one or more urine test strips.

13. The urine test method of claim 10, wherein the one or more urine test strips are configured to test for one or more of pregnancy, alcohol, an illicit or legal drug, blood in the urine, urine glucose, a disease, and a point of care concern.

14. The urine test method of claim 10, wherein the attachment device comprises a lure lock that is selectively coupled to one of a urinary catheter and a urinary bag or tubing.

15. The urine test method of claim 14, wherein the attachment device further comprises a conduit coupling the lure lock to the housing.

16. The urine test method of claim 10, wherein the attachment devices comprises one of a funnel structure and a syringe attachment structure.

17. The urine test method of claim 16, wherein the attachment device further comprises a conduit coupling the one of the funnel structure and the syringe attachment structure to the housing.

18. The urine test method of claim 10, wherein the urine test device further comprises a drain mechanism in fluid communication with the one or more internal chambers of the housing operable for selectively draining urine from the housing.
